(19) United States
(12) Patent Application Publication
(10) Pub. No.: US 2005/0013850 A1
(43) Pub. Date: Jan. 20, 2005

(54) Device to assist hyperhydrosis therapy

(76) Inventors: Jan K. Caers, Westerlo (BE); Koenraad De Boulle, Erpemere (BE)

Correspondence Address: STEPHEN DONOVAN;ALLERGAN, INC., T2-7H, 2525 Dupont Drive, Irvine, CA, 92612, US

(21) Appl. No.: 10/621,054

(22) Filed: Jul. 15, 2003

(51) Int. Cl. A61K 9/70

(52) U.S. Cl. 424/443

(57) ABSTRACT

A device to assist hyperhydrosis therapy is disclosed. The device can comprise a flexible, plastic oval with holes for placement over an area of skin which exhibits excessive sweat secretion. After placement of the device a pen or other mark is inserted through the holes to thereby easily establish a visible grid of where to inject an antihyperhydrotic pharmaceutical, such as a botulinum toxin.

### BACKGROUND

The present invention relates to a device for assisting hyperhydrosis therapy. In particular, the present invention relates to a dermal overlay device for assisting hyperhydrosis therapy.

Human sweat as part of a normal thermoregulation process. Additionally, sweating can be is a normal physiological response to a psychological stress or emotional stimuli. For most people, sweating is only a minor cosmetic annoyance. For others, however, sweating may be excessive and become a socially or medically crippling handicap. Hyperhidrosis is a disorder in which there is an exaggerated sweat secretion involving both the eccrine and the apocrine sweat glands. The excessive sweating usually occurs in the palms, soles, and axillae. Palmar hyperhidrosis is a condition of excessive sweating in the hand. Such condition may be socially embarrassing. Plantar hyperhidrosis is a condition of excessive sweating in the foot. This condition may cause blisters, infections, and bromohidrosis. Axillary hyperhidrosis is a condition of excessive sweating in the armpit. In axillary hyperhidrosis, as much as 26 mL/h of sweat can be excreted from each armpit. Such excessive sweating is not only socially embarrassing but may even cause staining and rotting of clothes.

Presently, the cause of hyperhidrosis is unknown. However, what is known is that the 3 to 4 million sweat glands of the body are under the control of the hypothalamus and the sympathetic system. Afferent impulses from sensors on the skin and other parts of the body travel to the hypothalamus, which integrates the information for chemoregulation of the body. The preoptic area of the anterior hypothalamus then sends efferent impulses via sympathetic fibers back out to the body. Segment T2 to T4 of the spinal chord innervate the head and neck area; fibers in segment T2 to T8 innervate the upper limbs; fibers in segment T6 to T10 innervate the trunk; and finally fibers in T11 to T12 innervate the lower extremities.

Although sympathetic innervations typically rely on adrenergic neurotransmitters, acetylcholine is the neurotransmitter released by the sympathetic nerve terminals involved in innervating the sweat glands. However, that is not to say that only acetylcholine can innervate the sweat glands. Some reports have shown that eccrine and apocrine glands respond to alpha.- and .beta.-adrenergic agonists as well.

Although the hypothalamus has a significant role in controlling the rate of sweating, other physical variables may affect the rate of sweat secretion. For example, sweating rate may also be affected by variables such as wetness and blood flow. Additionally, the rate of sweating varies greatly among people and is related to acclimatization, sex, age, and maybe even diet.

With respect to treating hyperhidrosis, various treatments are being used. For example, topical administration aluminum chloride is a common practice. It is thought that aluminum chloride mechanically obstruct eccrine sweat glands to reduce sweating, although some evidence shows that the reduction in sweat may result from atrophy of the secretory cells. A downside of using aluminum chloride is that the aluminum chloride may react with the water content of the sweat to form hydrochloric acid. The formation of hydrochloric acid may cause severe skin irritation.

Other topical preparations are also being used. For example, treatment of plantar and palmar hyperhidrosis includes use of glutaraldehyde and tannic acid (strong tea). However, this treatment may cause a browning of the skin.

Anticholinergics, both systemic and topical, are also being used. However, most patients cannot tolerate the side effects.

In addition to the described adverse effect of the above methods, the above treatment methods are effective to alleviate excessive sweating for only a brief duration of time, thus requiring frequent treatments, i.e. daily or weekly.

Surgical treatment involving sweat gland excision and sympathectomy may provide for a longer duration of alleviation from hyperhidrosis. However, these invasive treatments are rarely indicated due to the adverse consequences and cost. For example, surgery may cause contractures. Sympathectomy may result in complications including infection, pneumothorax, Horner's syndrome, resumption of sweating, and compensatory hyperhidrosis. Additionally, hyperhidrosis may resume after surgery or sympathectomy.

Subdermal injections of a botulinum toxin at the site of an excessive sweat secretion have been successfully used to treat hyperhydrosis. See e.g. Naumann M., Botulinum toxin type A in the treatment of focal hyperhidrosis, J. Cutaneous Laser Ther 2001;3(1):42-43, and; U.S. Pat. No. 5,766,605 (Sanders). Treatment typically entails, at each treatment session, making a number of injections into the hyperhydrotic skin, so as to achieve the desired distribution of the botulinum toxin into the target area, as opposed to making only one or a few injections. Typically, after determining the dimensions of a dermal area exhibiting of excessive sweat secretion, as by use of an iodine starch test, the attending physician attempts to indicate the locations of botulinum toxin injection by marking a pattern (a grid) of multiple spaced dots on the target skin area. Often the injection location dots are neither properly spaced nor of an appropriate number when such a freehand method is used. It is known to use a multiple injection plate for the treatment of hyperhydrosis wherein five or seven needles puncture the skin at the same time. Grimalt R., et al., Multi-injection plate for botulinum toxin application in the treatment of axillary hyperhidrosis, Dermatol Surg 2001 June;27(6):543-544.

Botulinum Toxin

The genus Clostridium has more than one hundred and twenty seven species, grouped according to their morphology and functions. The anaerobic, gram positive bacterium Clostridium botulinum produces a potent polypeptide neurotoxin, botulinum toxin, which causes a neuroparalytic illness in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs infected with a Clostridium botulinum culture or spores. The botulinum toxin can apparently pass unattenuated through the lining of the gut and attack peripheral motor neurons. Symptoms of botulinum toxin intoxication can progress from difficulty walking, swallowing, and speaking to paralysis of the respiratory muscles and death.

Botulinum toxin type A is the most lethal natural biological agent known to man. About 50 picograms of a commercially available botulinum toxin type A (purified neurotoxin complex)¹ is a LD₅₀ in mice
¹ Available from Allergan, Inc., of Irvine, Calif. under the tradename BOTOX® in 100 unit vials) (i.e. 1 unit). One unit of BOTOX® contains about 50 picograms (about 56 attomoles) of botulinum toxin type A complex. Interestingly, on a molar basis, botulinum toxin type A is about 1.8 billion times more lethal than diphtheria, about 600 million times more lethal than sodium cyanide, about 30 million times more lethal than cobra toxin and about 12 million times more lethal than cholera. Singh, Critical Aspects of Bacterial Protein Toxins, pages 63-84 (chapter 4) of Natural Toxins II, edited by B. R. Singh et al., Plenum Press, New York (1976) (where the stated LD₅₀ of botulinum toxin type A of 0.3 ng equals 1 U is corrected for the fact that about 0.05 ng of BOTOX® equals 1 unit). One unit (U) of botulinum toxin is defined as the LD₅₀ upon intraperitoneal injection into female Swiss Webster mice weighing 18 to 20 grams each.

Seven generally immunologically distinct botulinum neurotoxins have been characterized, these being respectively botulinum neurotoxin serotypes A, B, C₁, D, E, F and G each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD₅₀ for botulinum toxin type A. Moyer E et al., Botulinum Toxin Type B: Experimental and Clinical Experience, being chapter 6, pages 71-85 of “Therapy With Botulinum Toxin”, edited by Jankovic, J. et al. (1994), Marcel Dekker, Inc. Botulinum toxin apparently binds with high affinity to cholinergic motor neurons, is translocated into the neuron and blocks the release of acetylcholine. Additional uptake can take place through low affinity receptors, as well as by phagocytosis and pinocytosis.

Regardless of serotype, the molecular mechanism of toxin intoxication appears to be similar and to involve at least three steps or stages. In the first step of the process, the toxin binds to the presynaptic membrane of the target neuron through a specific interaction between the heavy chain, H chain, and a cell surface receptor; the receptor is thought to be different for each type of botulinum toxin and for tetanus toxin. The carboxyl end segment of the H chain, H_{C}, appears to be important for targeting of the toxin to the cell surface.

In the second step, the toxin crosses the plasma membrane of the poisoned cell. The toxin is first engulfed by the cell through receptor-mediated endocytosis, and an endosome containing the toxin is formed. The toxin then escapes the endosome into the cytoplasm of the cell. This step is thought to be mediated by the amino end segment of the H chain, H_{N}, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The toxin (or at a minimum the light chain) then translocates through the endosomal membrane into the cytoplasm.

The last step of the mechanism of botulinum toxin activity appears to involve reduction of the disulfide bond joining the heavy chain, H chain, and the light chain, L chain. The entire toxic activity of botulinum and tetanus toxins is contained in the L chain of the holotoxin; the L chain is a zinc (Zn++) endopeptidase which selectively cleaves proteins essential for recognition and docking of neurotransmitter-containing vesicles with the cytoplasmic surface of the plasma membrane, and fusion of the vesicles with the plasma membrane. Tetanus neurotoxin, botulinum toxin types B, D, F, and G cause degradation of synaptobrevin (also called vesicle-associated membrane protein (VAMP)), a synaptosomal membrane protein. Most of the VAMP present at the cytoplasmic surface of the synaptic vesicle is removed as a result of any one of these cleavage events. Botulinum toxin serotype A and E cleave SNAP-25. Botulinum toxin serotype C₁ was originally thought to cleave syntaxin, but was found to cleave syntaxin and SNAP-25. Each of the botulinum toxins specifically cleaves a different bond, except botulinum toxin type B (and tetanus toxin) which cleave the same bond. Each of these cleavages block the process of vesicle-membrane docking, thereby preventing exocytosis of vesicle content.

Botulinum toxins have been used in clinical settings for the treatment of neuromuscular disorders characterized by hyperactive skeletal muscles (i.e. motor disorders). In 1989 a botulinum toxin type A complex has been approved by the U.S. Food and Drug Administration for the treatment of blepharospasm, strabismus and hemifacial spasm. Subsequently, a botulinum toxin type A was also approved by the FDA for the treatment of cervical dystonia and for the treatment of glabellar lines, and a botulinum toxin type B was approved for the treatment of cervical dystonia. Non-type A botulinum toxin serotypes apparently have a lower potency and/or a shorter duration of activity as compared to botulinum toxin type A. Clinical effects of peripheral intramuscular botulinum toxin type A are usually seen within one week of injection. The typical duration of symptomatic relief from a single intramuscular injection of botulinum toxin type A averages about three months, although significantly longer periods of therapeutic activity have been reported.

Although all the botulinum toxins serotypes apparently inhibit release of the neurotransmitter acetylcholine at the neuromuscular junction, they do so by affecting different neurosecretory proteins and/or cleaving these proteins at different sites. For example, botulinum types A and E both cleave the 25 kiloDalton (kD) synaptosomal associated protein (SNAP-25), but they target different amino acid sequences within this protein. Botulinum toxin types B, D, F and G act on vesicle-associated protein (VAMP, also called synaptobrevin), with each serotype cleaving the protein at a different site. Finally, botulinum toxin type C₁ has been shown to cleave both syntaxin and SNAP-25. These differences in mechanism of action may affect the relative potency and/or duration of action of the various botulinum toxin serotypes. Apparently, a substrate for a botulinum toxin can be found in a variety of different cell types. See e.g. Biochem J 1;339 (pt 1):159-65:1999, and Mov Disord, 10(3):376:1995 (pancreatic islet B cells contains at least SNAP-25 and synaptobrevin).

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin type A complex can be produced by Clostridial bacterium as 900 kD, 500 kD and 300 kD forms. Botulinum toxin types B and C₁ is apparently produced as only a 700 kD or 500 kD complex. Botulinum toxin type D is produced as both 300 kD and 500 kD complexes. Finally, botulinum toxin types E and F are produced as only approximately 300 kD complexes. The complexes (i.e. molecular weight greater than about 150 kD) are believed to contain a non-toxin hemaglutinin protein and a non-toxin and non-toxic nonhemaglutinin protein. These two non-toxin proteins (which along with the botulinum toxin molecule comprise the relevant neurotoxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex.

In vitro studies have indicated that botulinum toxin inhibits potassium cation induced release of both acetylcholine and norepinephrine from primary cell cultures of brainstem tissue. Additionally, it has been reported that botulinum toxin inhibits the evoked release of both glycine and glutamate in primary cultures of spinal cord neurons and that in brain synaptosome preparations botulinum toxin inhibits the release of each of the neurotransmitters acetylcholine, dopamine, norepinephrine (Habermann E., et al., Tetanus Toxin and Botulinum A and C Neurotoxins Inhibit Noradrenaline Release From Cultured Mouse Brain, J Neurochem 51(2);522-527:1988) CGRP, substance P and glutamate (Sanchez-Prieto, J., et al., Botulinum Toxin A Blocks Glutamate Exocytosis From Guinea Pig Cerebral Cortical Synaptosomes, Eur J. Biochem 165;675-681:1897. Thus, when adequate concentrations are used, stimulus-evoked release of most neurotransmitters is blocked by botulinum toxin. See e.g. Pearce, L. B., Pharmacologic Characterization of Botulinum Toxin For Basic Science and Medicine, Toxicon 35(9);1373-1412 at 1393; Bigalke H., et al., Botulinum A Neurotoxin Inhibits Non-Cholinergic Synaptic Transmission in Mouse Spinal Cord Neurons in Culture, Brain Research 360;318-324:1985; Habermann E., Inhibition by Tetanus and Botulinum A Toxin of the release of [³H]Noradrenaline and [³H]GABA From Rat Brain Homogenate, Experientia 44;224-226:1988, Bigalke H., et al., Tetanus Toxin and Botulinum A Toxin Inhibit Release and Uptake of Various Transmitters, as Studied with Particulate Preparations From Rat Brain and Spinal Cord, Naunyn-Schmiedeberg's Arch Pharmacol 316;244-251:1981, and; Jankovic J. et al., Therapy With Botulinum Toxin, Marcel Dekker, Inc., (1994), page 5.

Botulinum toxin type A can be obtained by establishing and growing cultures of Clostridium botulinum in a fermenter and then harvesting and purifying the fermented mixture in accordance with known procedures. All the botulinum toxin serotypes are initially synthesized as inactive single chain proteins which must be cleaved or nicked by proteases to become neuroactive. The bacterial strains that make botulinum toxin serotypes A and G possess endogenous proteases and serotypes A and G can therefore be recovered from bacterial cultures in predominantly their active form. In contrast, botulinum toxin serotypes C₁, D and E are synthesized by nonproteolytic strains and are therefore typically unactivated when recovered from culture. Serotypes B and F are produced by both proteolytic and nonproteolytic strains and therefore can be recovered in either the active or inactive form. However, even the proteolytic strains that produce, for example, the botulinum toxin type B serotype only cleave a portion of the toxin produced. The exact proportion of nicked to unnicked molecules depends on the length of incubation and the temperature of the culture. Therefore, a certain percentage of any preparation of, for example, the botulinum toxin type B toxin is likely to be inactive, possibly accounting for the known significantly lower potency of botulinum toxin type B as compared to botulinum toxin type A. The presence of inactive botulinum toxin molecules in a clinical preparation will contribute to the overall protein load of the preparation, which has been linked to increased antigenicity, without contributing to its clinical efficacy. Additionally, it is known that botulinum toxin type B has, upon intramuscular injection, a shorter duration of activity and is also less potent than botulinum toxin type A at the same dose level.

High quality crystalline botulinum toxin type A can be produced from the Hall A strain of Clostridium botulinum with characteristics of ≧3×10⁷ U/mg, an A₂₆₀/A₂₇₈ of less than 0.60 and a distinct pattern of banding on gel electrophoresis. The known Shantz process can be used to obtain crystalline botulinum toxin type A, as set forth in Shantz, E. J., et al, Properties and use of Botulinum toxin and Other Microbial Neurotoxins in Medicine, Microbiol Rev. 56;80-99:1992. Generally, the botulinum toxin type A complex can be isolated and purified from an anaerobic fermentation by cultivating Clostridium botulinum type A in a suitable medium. The known process can also be used, upon separation out of the non-toxin proteins, to obtain pure botulinum toxins, such as for example: purified botulinum toxin type A with an approximately 150 kD molecular weight with a specific potency of 1-2×10⁸ LD₅₀ U/mg or greater; purified botulinum toxin type B with an approximately 156 kD molecular weight with a specific potency of 1-2×10⁸ LD₅₀ U/mg or greater, and; purified botulinum toxin type F with an approximately 155 kD molecular weight with a specific potency of 1-2×10⁷ LD₅₀ U/mg or greater.

Botulinum toxins and/or botulinum toxin complexes can be obtained from List Biological Laboratories, Inc., Campbell, California; the Centre for Applied Microbiology and Research, Porton Down, U.K.; Wako (Osaka, Japan), Metabiologics (Madison, Wis.) as well as from Sigma Chemicals of St Louis, Mo. Pure botulinum toxin can also be used to prepare a pharmaceutical composition.

As with enzymes generally, the biological activities of the botulinum toxins (which are intracellular peptidases) is dependant, at least in part, upon their three dimensional conformation. Thus, botulinum toxin type A is detoxified by heat, various chemicals surface stretching and surface drying. Additionally, it is known that dilution of the toxin complex obtained by the known culturing, fermentation and purification to the much, much lower toxin concentrations used for pharmaceutical composition formulation results in rapid detoxification of the toxin unless a suitable stabilizing agent is present. Dilution of the toxin from milligram quantities to a solution containing nanograms per milliliter presents significant difficulties because of the rapid loss of specific toxicity upon such great dilution. Since the toxin may be used months or years after the toxin containing pharmaceutical composition is formulated, the toxin can stabilized with a stabilizing agent such as albumin and gelatin.

A commercially available botulinum toxin containing pharmaceutical composition is sold under the trademark BOTOX® (available from Allergan, Inc., of Irvine, Calif.). BOTOX® consists of a purified botulinum toxin type A complex, albumin and sodium chloride packaged in sterile, vacuum-dried form. The botulinum toxin type A is made from a culture of the Hall strain of Clostridium botulinum grown in a medium containing N-Z amine and yeast extract. The botulinum toxin type A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 microns) prior to vacuum-drying. The vacuum-dried product is stored in a freezer at or below −5° C. BOTOX® can be reconstituted with sterile, non-preserved saline prior to intramuscular injection. Each vial of BOTOX® contains about 100 units (U) of Clostridium botulinum toxin type A purified neurotoxin complex, 0.5 milligrams of human serum albumin and 0.9 milligrams of sodium chloride in a sterile, vacuum-dried form without a preservative.

To reconstitute vacuum-dried BOTOX®, sterile normal saline without a preservative; (0.9% Sodium Chloride Injection) is used by drawing up the proper amount of diluent in the appropriate size syringe. Since BOTOX® may be denatured by bubbling or similar violent agitation, the diluent is gently injected into the vial. For sterility reasons BOTOX® is preferably administered within four hours after the vial is removed from the freezer and reconstituted. During these four hours, reconstituted BOTOX® can be stored in a refrigerator at about 20 C. to about 8° C. Reconstituted, refrigerated BOTOX® has been reported to retain its potency for at least about two weeks. Neurology, 48:249-53:1997.

It has been reported that botulinum toxin type A has been used in clinical settings as follows:
(1) about 75-125 units of BOTOX® per intramuscular injection (multiple muscles) to treat cervical dystonia;
(2) 5-10 units of BOTOX® per intramuscular injection to treat glabellar lines (brow furrows) (5 units injected intramuscularly into the procerus muscle and 10 units injected intramuscularly into each corrugator supercilii muscle);
(3) about 30-80 units of BOTOX® to treat constipation by intrasphincter injection of the puborectalis muscle;
(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid.
(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired).
(6) to treat upper limb spasticity following stroke by intramuscular injections of BOTOX® into five different upper limb flexor muscles, as follows:
(a) flexor digitorum profundus: 7.5 U to 30 U
(b) flexor digitorum sublimus: 7.5 U to 30 U
(c) flexor carpi ulnaris: 10 U to 40 U
(d) flexor carpi radialis: 15 U to 60 U
(e) biceps brachii: 50 U to 200 U. Each of the five indicated muscles has been injected at the same treatment session, so that the patient receives from 90 U to 360 U of upper limb flexor muscle BOTOX® by intramuscular injection at each treatment session.
(7) to treat migraine, pericranial injected (injected symmetrically into glabellar, frontalis and temporalis muscles) injection of 25 U of BOTOX® has showed significant benefit as a prophylactic treatment of migraine compared to vehicle as measured by decreased measures of migraine frequency, maximal severity, associated vomiting and acute medication use over the three month period following the 25 U injection.

Additionally, intramuscular botulinum toxin has been used in the treatment of tremor in patients with Parkinson's disease, although it has been reported that results have not been impressive. Marjama-Jyons, J., et al., Tremor-Predominant Parkinson's Disease, Drugs & Aging 16(4);273-278:2000.

It is known that botulinum toxin type A can have an efficacy for up to 12 months (European J. Neurology 6 (Supp 4): S111-S1150:1999), and in some circumstances for as long as 27 months, when used to treat glands, such as in the treatment of hyperhydrosis. See e.g. Bushara K., Botulinum toxin and rhinorrhea, Otolaryngol Head Neck Surg 1996;114(3):507, and The Laryngoscope 109:1344-1346:1999. However, the usual duration of an intramuscular injection of Botox® is typically about 3 to 4 months.

The success of botulinum toxin type A to treat a variety of clinical conditions has led to interest in other botulinum toxin serotypes. Two commercially available botulinum type A preparations for use in humans are BOTOX® available from Allergan, Inc., of Irvine, Calif., and Dysport® available from Beaufour Ipsen, Porton Down, England. A Botulinum toxin type B preparation (MyoBloc®) is available from Elan Pharmaceuticals of San Francisco, Calif.

In addition to having pharmacologic actions at the peripheral location, botulinum toxins may also have inhibitory effects in the central nervous system. Work by Weigand et al, Nauny-Schmiedeberg's Arch. Pharmacol. 1976; 292,161-165, and Habermann, Nauny-Schmiedeberg's Arch. Pharmacol. 1974; 281, 47-56 showed that botulinum toxin is able to ascend to the spinal area by retrograde transport. As such, a botulinum toxin injected at a peripheral location, for example intramuscularly, may be retrograde transported to the spinal cord.

A botulinum toxin has also been proposed for the treatment of rhinorrhea (chronic discharge from the nasal mucous membranes, i.e. runny nose), rhinitis (inflammation of the nasal mucous membranes), hyperhydrosis and other disorders mediated by the autonomic nervous system (U.S. Pat. No. 5,766,605), tension headache, (U.S. Pat. No. 6,458,365), migraine headache (U.S. Pat. No. 5,714,468), post-operative pain and visceral pain (U.S. Pat. No. 6,464,986), pain treatment by intraspinal toxin administration (U.S. Pat. No. 6,113,915), Parkinson's disease and other diseases with a motor disorder component, by intracranial toxin administration (U.S. Pat. No. 6,306,403), hair growth and hair retention (U.S. Pat. No. 6,299,893), psoriasis and dermatitis (U.S. Pat. No. 5,670,484), injured muscles (U.S. Pat. No. 6,423,319, various cancers (U.S. Pat. No. 6,139,845), pancreatic disorders (U.S. Pat. No. 6,143,306), smooth muscle disorders (U.S. Pat. No. 5,437,291, including injection of a botulinum toxin into the upper and lower esophageal, pyloric and anal sphincters)), prostate disorders (U.S. Pat. No. 6,365,164), inflammation, arthritis and gout (U.S. Pat. No. 6,063,768), juvenile cerebral palsy (U.S. Pat. No. 6,395,277), inner ear disorders (U.S. Pat. No. 6,265,379), thyroid disorders (U.S. Pat. No. 6,358,513), parathyroid disorders (U.S. Pat. No. 6,328,977) and neurogenic inflammation (U.S. Pat. No. 6,063,768). Additionally, controlled release toxin implants are known (see e.g. U.S. Pat. Nos. 6,306,423 and 6,312,708).

Tetanus toxin, as wells as derivatives (i.e. with a non-native targeting moiety), fragments, hybrids and chimeras thereof can also have therapeutic utility. The tetanus toxin bears many similarities to the botulinum toxins. Thus, both the tetanus toxin and the botulinum toxins are polypeptides made by closely related species of Clostridium (Clostridium tetani and Clostridium botulinum, respectively). Additionally, both the tetanus toxin and the botulinum toxins are dichain proteins composed of a light chain (molecular weight about 50 kD) covalently bound by a single disulfide bond to a heavy chain (molecular weight about 100 kD). Hence, the molecular weight of tetanus toxin and of each of the seven botulinum toxins (non-complexed) is about 150 kD. Furthermore, for both the tetanus toxin and the botulinum toxins, the light chain bears the domain which exhibits intracellular biological (protease) activity, while the heavy chain comprises the receptor binding (immunogenic) and cell membrane translocational domains.

Further, both the tetanus toxin and the botulinum toxins exhibit a high, specific affinity for gangliocide receptors on the surface of presynaptic cholinergic neurons. Receptor mediated endocytosis of tetanus toxin by peripheral cholinergic neurons results in retrograde axonal transport, blocking of the release of inhibitory neurotransmitters from central synapses and a spastic paralysis. Contrarily, receptor mediated endocytosis of botulinum toxin by peripheral cholinergic neurons results in little if any retrograde transport, inhibition of acetylcholine exocytosis from the intoxicated peripheral motor neurons and a flaccid paralysis.

Finally, the tetanus toxin and the botulinum toxins resemble each other in both biosynthesis and molecular architecture. Thus, there is an overall 34% identity between the protein sequences of tetanus toxin and botulinum toxin type A, and a sequence identity as high as 62% for some functional domains. Binz T. et al., The Complete Sequence of Botulinum Neurotoxin Type A and Comparison with Other Clostridial Neurotoxins, J Biological Chemistry 265(16);9153-9158:1990.

Acetyicholine

Typically only a single type of small molecule neurotransmitter is released by each type of neuron in the mammalian nervous system, although there is evidence which suggests that several neuromodulators can be released by the same neuron. The neurotransmitter acetylcholine is secreted by neurons in many areas of the brain, but specifically by the large pyramidal cells of the motor cortex, by several different neurons in the basal ganglia, by the motor neurons that innervate the skeletal muscles, by the preganglionic neurons of the autonomic nervous system (both sympathetic and parasympathetic), by the bag 1 fibers of the muscle spindle fiber, by the postganglionic neurons of the parasympathetic nervous system, and by some of the postganglionic neurons of the sympathetic nervous system. Essentially, only the postganglionic sympathetic nerve fibers to the sweat glands, the piloerector muscles and a few blood vessels are cholinergic as most of the postganglionic neurons of the sympathetic nervous system secret the neurotransmitter norepinephine. In most instances acetylcholine has an excitatory effect. However, acetylcholine is known to have inhibitory effects at some of the peripheral parasympathetic nerve endings, such as inhibition of heart rate by the vagal nerve.

The efferent signals of the autonomic nervous system are transmitted to the body through either the sympathetic nervous system or the parasympathetic nervous system. The preganglionic neurons of the sympathetic nervous system extend from preganglionic sympathetic neuron cell bodies located in the intermediolateral horn of the spinal cord. The preganglionic sympathetic nerve fibers, extending from the cell body, synapse with postganglionic neurons located in either a paravertebral sympathetic ganglion or in a prevertebral ganglion. Since, the preganglionic neurons of both the sympathetic and parasympathetic nervous system are cholinergic, application of acetylcholine to the ganglia will excite both sympathetic and parasympathetic postganglionic neurons.

Acetylcholine activates two types of receptors, muscarinic and nicotinic receptors. The muscarinic receptors are found in all effector cells stimulated by the postganglionic, neurons of the parasympathetic nervous system as well as in those stimulated by the postganglionic cholinergic neurons of the sympathetic nervous system. The nicotinic receptors are found in the adrenal medulla, as well as within the autonomic ganglia, that is on the cell surface of the postganglionic neuron at the synapse between the preganglionic and postganglionic neurons of both the sympathetic and parasympathetic systems. Nicotinic receptors are also found in many nonautonomic nerve endings, for example in the membranes of skeletal muscle fibers at the neuromuscular junction.

Acetylcholine is released from cholinergic neurons when small, clear, intracellular vesicles fuse with the presynaptic neuronal cell membrane. A wide variety of non-neuronal secretory cells, such as, adrenal medulla (as well as the PC12 cell line) and pancreatic islet cells release catecholamines and parathyroid hormone, respectively, from large dense-core vesicles. The PC12 cell line is a clone of rat pheochromocytoma cells extensively used as a tissue culture model for studies of sympathoadrenal development. Botulinum toxin inhibits the release of both types of compounds from both types of cells in vitro, permeabilized (as by electroporation) or by direct injection of the toxin into the denervated cell. Botulinum toxin is also known to block release of the neurotransmitter glutamate from cortical synaptosomes cell cultures.

A neuromuscular junction is formed in skeletal muscle by the proximity of axons to muscle cells. A signal transmitted through the nervous system results in an action potential at the terminal axon, with activation of ion channels and resulting release of the neurotransmitter acetylcholine from intraneuronal synaptic vesicles, for example at the motor endplate of the neuromuscular junction. The acetylcholine crosses the extracellular space to bind with acetylcholine receptor proteins on the surface of the muscle end plate. Once sufficient binding has occurred, an action potential of the muscle cell causes specific membrane ion channel changes, resulting in muscle cell contraction. The acetylcholine is then released from the muscle cells and metabolized by cholinesterases in the extracellular space. The metabolites are recycled back into the terminal axon for reprocessing into further acetylcholine.

What is needed therefore is a method for facilitating hyperhydrosis therapy by assisting the marking of a target skin area with a grid or pattern of injection location marks or dots, at which locations (i.e. at the dots) an antihyperhydrotic pharmaceutical, such as a botulinum toxin can be injected.

### SUMMARY

The present invention meets this need and provides needed a device for facilitating hyperhydrosis therapy. The device can be used to assist marking of a target skin area with a grid or pattern of injection location marks or dots, at which locations (i.e. at the dots) an antihyperhydrotic pharmaceutical, such as a botulinum toxin can be injected.

The botulinum toxin (as either a complex (i.e. about 300 to about 900 kDa] or as a pure [i.e. about 150 kDa molecule] used can be a botulinum toxin A, B, C, D, E, F or G.

As used herein “about” means approximately or nearly and in the context of a numerical value or range set forth herein means±10% of the numerical value or range recited or claimed.

A device for assisting hyperhydrosis therapy can comprise a material with an upper face and a lower face. The lower face of the material is suitable for placement in contact with an area of the dermis of a patient with hyperhydrosis. The dermal area is an area which exhibits excessive sweat secretion. The material can have a plurality of perforations which extend completely through the material from the upper face to the lower face.

Additionally, the material can have an exterior border which circumscribes the material. The exterior border is not perforated because a user presses down on the border to hold the device in place when it is in use.

Preferably, the material is flexible, so that when the material is pressed again the dermal area, substantially all of the exterior border is in contact with the dermal area. The perforations in the material can be spaced apart by a first uniform distance. The device can also comprise a second plurality of perforations spaced apart by a second uniform distance. The first uniform distance is not equal to the second uniform distance.

At least one (and as many as all) of the perforations can have a bore with a first end opening at the upper face and a second end opening at the lower face, wherein the diameter of the first end of the bore is greater than the diameter of the second end of the bore.

A method for assisting a hyperhydrosis therapy through use of our device can have the steps of: determining a dermal area of a patient which exhibits hyperhydrosis; placing in contact with the dermal area the lower face of the device comprising; extending a marker through a perforation so as to mark a dermal surface under the lower face of the material, and; removing the device from contact with the dermal area.

The determining step can be by use of an iodine starch test. This method can further comprise after the removing step, the step of injecting a botulinum toxin at the location of the mark on the dermal area.

### DRAWINGS

The following drawings are provided to assist understanding of aspects and features of the present invention.

FIG. 1 is top view of an embodiment of a device for assisting hyperhydrosis therapy within the scope of the present invention, showing a plurality of perforations in the device.

FIG. 2 is a is top view of a second embodiment of a device for assisting hyperhydrosis therapy within the scope of the present invention, showing a plurality of more closely set perforations.

FIG. 3 is a is top view of a second embodiment of a device for assisting hyperhydrosis therapy within the scope of the present invention, showing a plurality of two different set of perforations.

FIG. 4 is an enlarged side cross sectional view through one of a perforations in the device of FIGS. 1, 2 or 3.

### DESCRIPTION

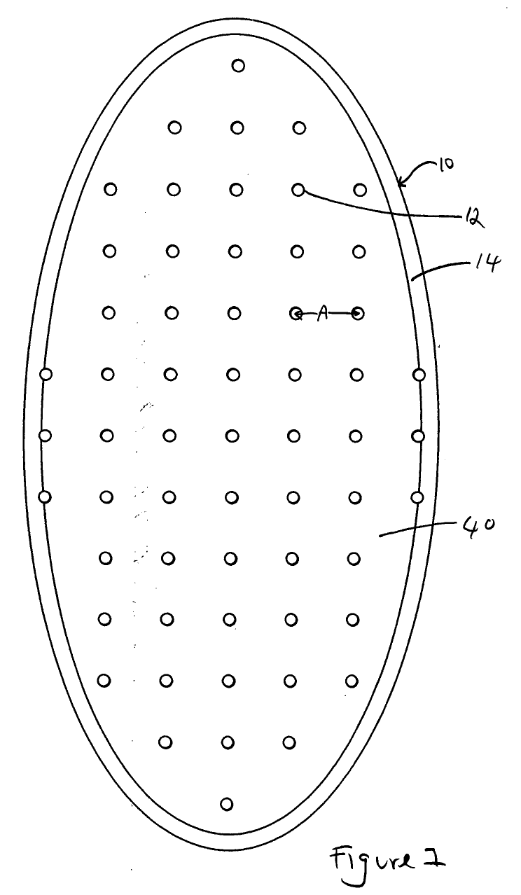

The present invention is based on the discovery that hyperhydrosis therapy can be assisted by use of a dermal overlay device. As shown by FIG. 1, an embodiment of our invention can be a device 10 comprised of a material, such as a flexible plastic, suitable (i.e. no sharp protrusions, non-irritating) for firm, though temporary, placement against a patch or area of hyperhydrotic skin of a hyperhydrosis patient. The device 10 can be made of a transparent material and has a plurality of through holes or perforations 12. A border 14 circumscribes the device 10. Preferably, the perforations 12 are separated by a uniform distance A, so as to facilitate an even distribution of an injected antihyperhydrotic pharmaceutical. The distance A can be about 2 cm.

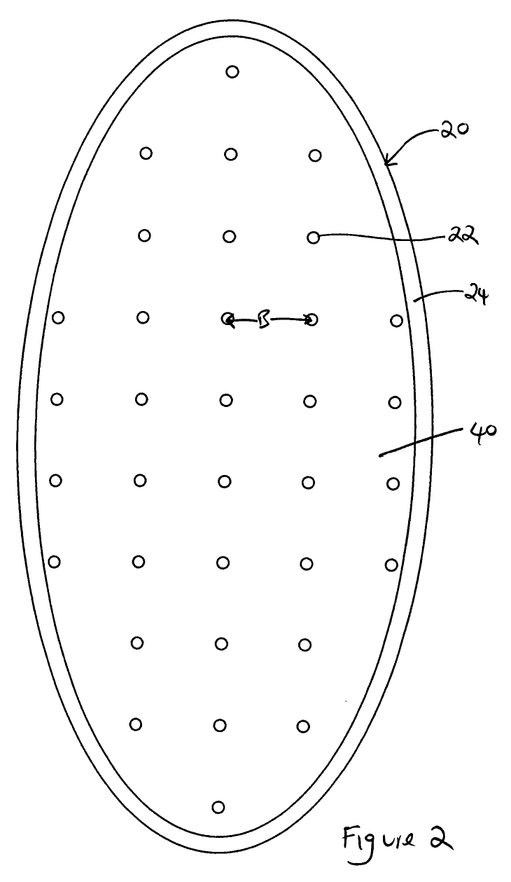

An alternate embodiment of our invention, as shown by FIG. 2, can comprise a device 20 comprised of a material, such as a bendable plastic, suitable (i.e. smooth, not irritating upon transient skin contact) for firm, though temporary, placement against a patch or area of hyperhydrotic skin of a hyperhydrosis patient. The device 20 can be made of a transparent material and has a plurality of through holes or perforations 22. A border 24 circumscribes the device 20. Preferably, the perforations 22 are separated by a uniform distance B, so as to facilitate an even distribution of an injected antihyperhydrotic pharmaceutical. The distance B can be about 1.5 cm.

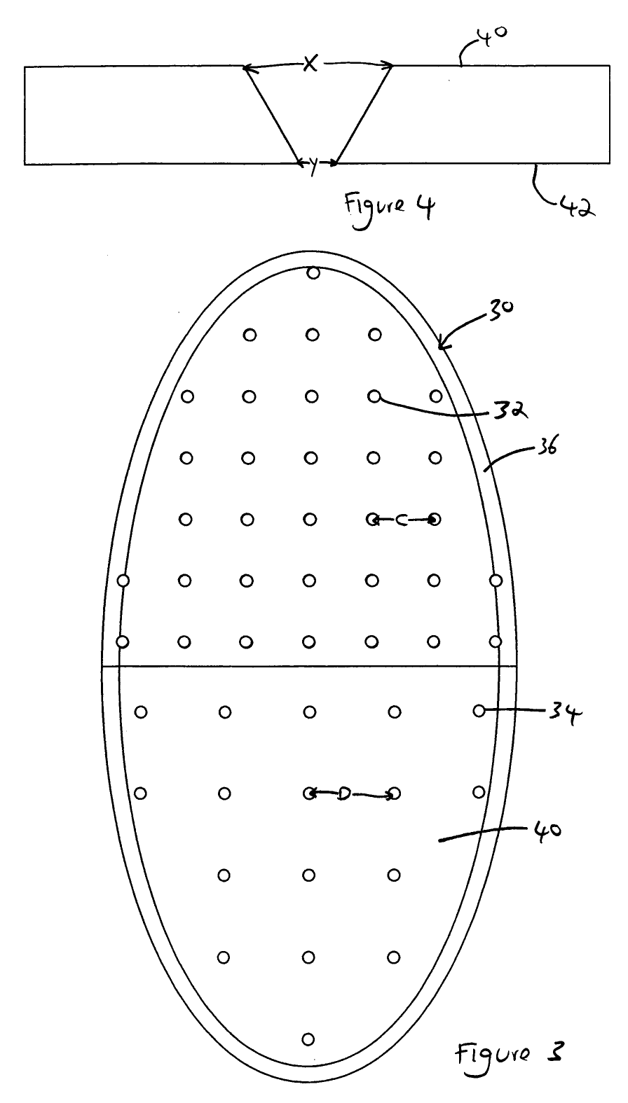

A third alternate embodiment of our invention, as shown by FIG. 3, can comprise a device 30 comprised of a material, such as a flexible plastic, suitable (i.e. no sharp protrusions, non-irritating) for firm, though temporary, placement against a patch or area of hyperhydrotic skin of a hyperhydrosis patient. The device 30 can be made of a transparent material and has a plurality of a first set of holes or perforations 32 and a second set of holes or perforations 34. A border 36 can circumscribe the device 30. Preferably, the perforations 32 are separated by a uniform distance C, so as to facilitate an even distribution of an injected antihyperhydrotic pharmaceutical. The perforations 34 can be separated by a uniform distance D, so as to facilitate an even distribution of an injected antihyperhydrotic pharmaceutical with a different injection density (i.e. C is not equal to D). The distance between the perforations 12, 22, 32 or 34 can be between about 0.1 cm to about 4 cm.

As shown by FIG. 4 which is an enlarged, side cross sectional view through one of the perforations of FIGS. 1, 2 or 3, the device has a top face 40 and a bottom face 42. A perforation can have a first end which opens onto the top face 40, which first end has a diameter X. The perforation can also have a second end which opens onto the bottom 42, which first end has a diameter Y. Preferably, and as illustrated by FIG. 4, diameter Y is less than diameter X, so that the bore of the perforation can have a conical shape. Such a conical shape is a preferred configuration for a bore of a perforation of the device because upon insertion of a marker such as a ball point pen into the first end of the perforation and through to the second end of the perforation (while the lower face of the device is in contact with and being pressed against the skin of a patient), the marker will be held firmly in the perforation and will make a point mark or dot on the skin of the patient. Since the device has multiple such perforations, rapid and accurate use of the marker to mark a series of dots onto the skin of the patient is thereby assisted.

In practice the devise can be used by placing the lower face of the device against an area of target skin (which can be, for example, hyperhydrotic axial (i.e. armpit), plantar or plamar skin) which has previously been determined to be an area of hyperhydrotic skin, as by observation or by use of a diagnosed test such as the iodine starch test. Thus, before treating focal hyperhidrosis, it can be necessary to find out what specific area of the body is producing excess sweat. This can be done using a diagnostic procedure known as the Minor or iodine starch test. For this test, a weak solution of iodine is applied to the skin. Then, powdered starch is dusted over the dried iodine. As the patient sweats, the areas where excessive sweating occurs are stained a bluish color by the iodine starch mixture, thereby showing where the sweat glands are overactive. Gravimetry is another test which measures exactly how much a patient sweats. In gravimetry blotting paper is pressed against the skin to soak up the sweat. Then, the blotting paper is weighed with a delicate scale to determine how much sweat has been absorbed.

The device is pressed against the skin (by pressing down on the border) and a marker is inserted into each of the perforations in turn. The device in then removed form contact with the skin leaving a grid pattern of dots on the skin showing where to inject the botulinum toxin.

The material which comprises the device can be a plastic, silicone or other suitable material. The material can be flexible and can be shaped and sized so as to follow the contours of an armpit, foot or hand where it can be applied.

Examples of botulinum toxins within the scope of the present invention include the botulinum toxin types A, B, C, D, E, F, and G.

Botulinum toxins for use according to the present invention can be stored in lyophilized, vacuum dried form in containers under vacuum pressure or as stable liquids. Prior to lyophilization the botulinum toxin can be combined with pharmaceutically acceptable excipients, stabilizers and/or carriers, such as albumin. The lyophilized material can be reconstituted with saline or water to create a solution or composition containing the botulinum toxin to be administered to the patient.

### EXAMPLE

The following non-limiting example sets forth a specific preferred method to use a device within the scope of the present invention and is not intended to limit the scope of the our invention.

### Example 1

### Use of Device for Assisting Hyperhydrosis Therapy

A female patient, 32 years old, is diagnosed through observation and use of the iodine starch with axial hyperhydrosis, in both armpits. The lower side of the device shown in FIG. 1 is pressed firmly against her left armpit (while her left arm is raised above her head) and a ball point pen is inserted into each of the perforations of the device in turn. The device is removed, leaving a clear grid pattern of dots on her arm pit. The same procedure is followed for the right armpit. A botulinum toxin in then injected at the site of each dot, thereby treating her hyperhydrosis.

Although the present invention has been described in detail with regard to certain preferred methods, other embodiments, versions, and modifications within the scope of the present invention are possible. For example, the disclosed device can be made from various materials and in various shapes, with different perforations spacings and different perforation bore diameters.

All references, articles, patents, applications and publications set forth above are incorporated herein by reference in their entireties.

Accordingly, the spirit and scope of the following claims should not be limited to the descriptions of the preferred embodiments set forth above.

1. A device for assisting hyperhydrosis therapy, the device comprising:
(a) a material with an upper face and a lower face, the lower face of the material being suitable for placement in contact with an area of the dermis of a patient with hyperhydrosis, wherein the dermal area is an area which exhibits excessive sweat secretion, and;
(b) the material has a plurality of perforations which extend completely through the material from the upper face to the lower face.

2. The device of claim 1, wherein the material has an exterior border which circumscribes the material and wherein the exterior border is not perforated.

3. The device of claim 2, wherein the material is flexible, so that when the material is pressed again the dermal area, substantially all of the exterior border is in contact with the dermal area.

4. The device of claim 1 wherein the perforations are spaced apart by a first uniform distance.

5. The device of claim 4, where the device further comprises a second plurality of perforations spaced apart by a second uniform distance, and wherein the first uniform distance is not equal to the second uniform distance.

6. The method of claim 1, wherein at least one of the perforations has a bore with a first end opening at the upper face and a second end opening at the lower face, wherein the diameter of the first end of the bore is greater than the diameter of the second end of the bore.

7. The method of claim 6, wherein all the perforations have a bore with a first end opening at the upper face and a second end opening at the lower face, and wherein the diameter of the first end of each bore of each perforation is greater than the diameter of the second end of the bore.

8 A device for assisting hyperhydrosis therapy, the device comprising:
(a) a material with an upper face and a lower face, the lower face of the material being suitable for placement in contact with an area of the dermis of a patient with hyperhydrosis, wherein the dermal area is an area which exhibits excessive sweat secretion;
(b) the material has a plurality of perforations which extend completely through the material from the upper face to the lower face, and;
(c) the material has an exterior border which circumscribes the material and wherein the exterior border is not perforated.

9. The device of claim 8, wherein the material is flexible, so that when the material is pressed again the dermal area, substantially all of the exterior border is in contact with the dermal area.

10. The device of claim 8 wherein the perforations are spaced apart by a first uniform distance.

11. The device of claim 10, where the device further comprises a second plurality of perforations spaced apart by a second uniform distance, and wherein the first uniform distance is not equal to the second uniform distance.

12. The device of claim 8, wherein at least one of the perforations has a bore with a first end opening at the upper face and a second end opening at the lower face, wherein the diameter of the first end of the bore is greater than the diameter of the second end of the bore.

13. The device of claim 12, wherein all the perforations have a bore with a first end opening at the upper face and a second end opening at the lower face, and wherein the diameter of the first end of each bore of each perforation is greater than the diameter of the second end of the bore.

14. A method for assisting a hyperhydrosis therapy, the method comprising:
(a) determining a dermal area of a patient which exhibits hyperhydrosis;
(b) placing in contact with the dermal area a lower face of a device comprising: (i) a material with an upper face and a lower face, and; (ii) the material having a plurality of perforations which extend completely through the material from the upper face to the lower face;
(c) extending a marker through a perforation so as to mark a dermal surface under the lower face of the material, and;
(d) removing the device from contact with the dermal area.

15. The method of claim 14, wherein the determining step comprises use of an iodine starch test.

16. The method of claim 14, further comprising after the removing step, the step of injecting a botulinum toxin at the location of the mark on the dermal area.

17. A method for assisting a hyperhydrosis therapy, the method comprising:
(a) determining a dermal area of a patient which exhibits hyperhydrosis;
(b) placing in contact with the dermal area a lower face of a device comprising: (i) a material with an upper face and a lower face, and; (ii) the material having a plurality of perforations which extend completely through the material from the upper face to the lower face;
(c) extending a marker through a perforation so as to mark a dermal surface under the lower face of the material;
(d) removing the device from contact with the dermal area, and;
(e) injecting a botulinum toxin at the location of the mark on the dermal area.
